SEQUENCE LISTING (2 sequences)

SEQ ID NO: 1 - protein, 281 aa, Artificial Sequence
  Description of Artificial Sequence Synthetic polypeptide
MQWNSTTFHQTLQDPRVRALYFPAGGSSSGTVSPAQNTVSAISSILSKTGDPVPNMENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

SEQ ID NO: 2 - protein, 226 aa, Artificial Sequence
  Description of Artificial Sequence Synthetic polypeptide
MENIASGLLGPLLVLQAGFFLLTKILTIPQSLDSWWTSLNFLGGTPVCLGQNSQSQISSHSPTCCPPICPGYRWMCLRRFIIFLCILLLCLIFLLVLLDYQGMLPVCPLIPGSSTTSTGPCKTCTTPAQGTSMFPSCCCTKPTDGNCTCIPIPSSWAFAKYLWEWASVRFSWLSLLVPFVQWFVGLSPTVWLSVIWMMWYWGPSLYNILSPFMPLLPIFFCLWVYI

(19) United States
(12) Patent Application Publication
(10) Pub. No.: US 2009/0280474 A1
(43) Pub. Date: Nov. 12, 2009

(54) METHOD FOR DETECTING A VIRUS

(75) Inventors: Lynn A. Martin, Lindenhurst, IL (US); Sushil G. Devare, Northbrook, IL (US); Mary C. Kuhns, Lincolnshire, IL (US)

Correspondence Address: PAUL D. YASGER;ABBOTT LABORATORIES, 100 ABBOTT PARK ROAD, DEPT. 377/AP6A, ABBOTT PARK, IL, 60064-6008, US

(73) Assignee: Abbott Laboratories, Abbott Park, IL (US)

(21) Appl. No.: 12/117,550

(22) Filed: May 8, 2008

(51) Int. Cl. C12Q 1/70

(52) U.S. Cl. 435/5

(57) ABSTRACT

This invention is related a method for increasing the sensitivity of detecting a viral target in a sample. The sensitivity may be increased by disrupting a complex comprising the target or by measuring the level of the target from a larger volume of the sample.

### FIELD OF THE INVENTION

This invention relates to a method for detecting a viral target with increased sensitivity.

### BACKGROUND OF THE INVENTION

The hepatitis B virus (HBV) is estimated to have infected over 2 billion people worldwide. HBV is known to cause a variety of disease states from mild subclinical infection to chronic active and fulminant hepatitis. Over 400 million people, especially children and the elderly, are chronically infected with HBV. The hepatitis B virus is 100 times more infectious than the AIDS virus, yet it can be prevented with vaccination. A key strategy in controlling HBV infection is universal vaccination as well as early detection and treatment of infected individuals. Accordingly, HBV diagnostic assays have focused on improved and accurate detection of HBV viral antigens.

In order to improve HBV detection, reagents such as monoclonal antibodies, recombinant antigens, and detection reagents have been developed. Moreover, label-to-antibody or -antigen incorporation ratios can be increased in order to generate additional signal. However, this leads to high background levels, decreased sensitivity, higher initial reactive rates, and nonspecific binding. In order to maximize blood screening immunoassay quality, specificity should be maintained and false positive reactive rates should be minimized. Nucleic acid testing offers a particularly sensitive method for detecting HBV. However, commercial tests frequently incorporate sample extraction procedures and they require amplification of target or HBV DNA prior to detection. In contrast, immunoassays aimed at detecting HBV surface antigen (HBsAg) do not rely on sample extraction procedures and do not require any target DNA amplification. But the presence of HBV surface antibody (anti-HBs) in samples containing low levels of HBsAg may make it difficult or impossible to detect HBV. Accordingly, HBV infections can go undiagnosed in acute hepatitis, chronic HBV carriers or patients with occult HBV infection (OBI). Therefore, there is a need in the art for HBV immunoassays with improved sensitivity.

### SUMMARY OF THE INVENTION

Provided herein is a method for detecting a target in a sample, wherein the target may be bound to a first binding partner. The method may comprise (a) providing a sample, (b) displacing the target/first binding partner complex, and (c) contacting the sample with a second binding partner. The presence of a target/second binding partner complex may be indicative of the presence of the target in the sample. The method may further comprise detecting the target/second binding partner complex. Steps (b) and (c) may be performed concurrently, and may be performed on the sample in at least duplicate.

The first and second binding partners may each be an antibody, and the target may be a protein. The second binding partner may bind to an antigen of the target. The antigen may be a linear epitope, and may be a surface antigen or a core antigen. The antigen may be of a virus, which may be a hepatitis B virus, a hepatitis C virus, or a human immunodeficiency virus. The sample may be isolated from a patient with acute or chronic hepatitis B virus infection, and may also be isolated from a patient with occult hepatitis B virus infection. The sample may have a volume of at least 250 μL.

The displacing may be performed by decreasing the sample pH to 2-6, which may be with a solution comprising glycine at a concentration of 50 mM to 1M, and the pH of the solution may be from 1-2. The displacing may also be performed by increasing the temperature of the sample to 60° C. to 110° C., or by adding an agent capable of reducing disulfide bonds. The displaced first binding partner may be removed from the sample, which may be by using an antibody binding reagent. The antibody binding reagent may be anti-human Ig, and may be attached to a microparticle.

### BRIEF DESCRIPTION OF THE DRAWINGS

FIG. 1 shows that the sensitivity of an HBV detection assay to HBsAg is very similar between treated (low pH) compared to untreated (PBS) samples.

### DETAILED DESCRIPTION

Viral infections can be diagnosed by detecting the presence of a viral protein. However, it can be difficult to detect viral proteins when a patient has mounted an immune response to the virus. When the viral protein is bound in an immune complex, an antigen that would normally be bound by a detection antibody can be masked and therefore harder to detect. The decrease in detection is particularly more pronounced when the patient has a low-level infection. In this scenario, much of what little viral antigen there is in the sample may be unavailable for binding and detection using a standard antibody-based approach. Antibodies from a patient's immune response can also reduce detection of viral antigens when the patient has a chronic or acute infection.

The inventors have made the surprising discovery that improved sensitivity and detection of HBV can be achieved by displacing hepatitis B viral target proteins from anti-HBV surface antigen immune complexes prior to detecting the target antigen. Sensitivity of detection can also be enhanced by using increased volumes of sample and detecting the target antigen in at least duplicate samples. These approaches enable better detection of low levels of HBV such as low level viremia or in patients suffering from an occult HBV infection. Occult HBV infections can result from several factors, including the presence of anti-HBs antibodies, persistent low-level viremia, or the presence of HBV escape mutants and these infections can go undetected using current HBsAg assays.

The same principles of target antigen displacement can also be used to improve the sensitivity of detecting other targets that may be bound in a complex. For example, the detection of nucleic acids, nucleoproteins, and polypeptides can be improved by displacing them.

### 1. DEFINITIONS

The terminology used herein is for the purpose of describing particular embodiments only and is not intended to be limiting. As used in the specification and the appended claims, the singular forms “a,” “an” and “the” include plural referents unless the context clearly dictates otherwise.

For recitation of numeric ranges herein, each intervening number there between with the same degree of precision is explicitly contemplated. For example, for the range of 6-9, the numbers 7 and 8 are contemplated in addition to 6 and 9, and for the range 6.0-7.0, the numbers 6.0, 6.1, 6.2, 6.3, 6.4, 6.5, 6.6, 6.7, 6.8, 6.9, and 7.0 are explicitly contemplated.

a. Antibody

“Antibody” as used herein may mean an antibody of classes IgG, IgM, IgA, IgD or IgE, or fragments or derivatives thereof, including Fab, F(ab′)2, Fd, and single chain antibodies, diabodies, bispecific antibodies, bifunctional antibodies and derivatives thereof. The antibody may be a monoclonal antibody, polyclonal antibody, affinity purified antibody, or mixtures thereof which exhibits sufficient binding specificity to a desired epitope or a sequence derived therefrom. The polyclonal antibody may be of mammalian origin, such as human, goat, rabbit, or sheep. The antibody may also be a chimeric antibody. The antibody may be derivatized by the attachment of one or more chemical, peptide, or polypeptide moieties known in the art. The antibody may be conjugated with a chemical moiety. The antibody may be a specific binding member.

b. Attached

“Attached” or “immobilized” as used herein to refer to a polypeptide and a solid support may mean that the binding between the polypeptide and the solid support is sufficient to be stable under conditions of binding, washing, analysis, and removal. The binding may be covalent or non-covalent. Covalent bonds may be formed directly between the polypeptide and the solid support or may be formed by a cross linker or by inclusion of a specific reactive group on either the solid support or the probe or both molecules. Non-covalent binding may be one or more of electrostatic, hydrophilic, and hydrophobic interactions. Included in non-covalent binding is the covalent attachment of a molecule, such as streptavidin, to the support and the non-covalent binding of a biotinylated polypeptide to the streptavidin. Immobilization may also involve a combination of covalent and non-covalent interactions.

c. Epitope

“Epitope” or “antigen” as used herein may mean an antigenic determinant of a polypeptide. An epitope may comprise 3 amino acids in a spatial conformation which is unique to the epitope. An epitope may comprise at least 5, 6, 7, 8, 9, or 10 amino acids. Methods of examining spatial conformation are known in the art and include, X-ray crystallography and two-dimensional nuclear magnetic resonance. The antigen may also be a linear epitope. The antigen may be recombinant or synthetic.

d. Fragment

“Fragment” as used herein may mean a portion of a reference peptide or polypeptide.

e. Identical

“Identical” or “identity” as used herein in the context of two or more polypeptide sequences, may mean that the sequences have a specified percentage of residues that are the same over a specified region. The percentage may be calculated by optimally aligning the two sequences, comparing the two sequences over the specified region, determining the number of positions at which the identical residue occurs in both sequences to yield the number of matched positions, dividing the number of matched positions by the total number of positions in the specified region, and multiplying the result by 100 to yield the percentage of sequence identity. In cases where the two sequences are of different lengths or the alignment produces one or more staggered ends and the specified region of comparison includes only a single sequence, the residues of single sequence are included in the denominator but not the numerator of the calculation.

f. Indicator Reagent

“Indicator reagent” as used herein may be a composition comprising a label, which is capable of generating a measurable signal that is detectable by external means, and which may be conjugated or attached to a specific binding member for a particular polypeptide. The indicator reagent may be an antibody member of a specific binding pair for a particular polypeptide. The indicator reagent may also be a member of any specific binding pair, including hapten-anti-hapten systems such as biotin or anti-biotin, avidin, or biotin, a carbohydrate or a lectin, a complementary nucleotide sequence, an effector or a receptor molecule, an enzyme cofactor and an enzyme, or an enzyme inhibitor and an enzyme.

g. Label

“Label” or “detectable label” as used herein may mean a moiety capable of generating a signal that allows the direct or indirect quantitative or relative measurement of a molecule to which it is attached. The label may be a solid such as a microtiter plate, particle, microparticle, or microscope slide; an enzyme; an enzyme substrate; an enzyme inhibitor; coenzyme; enzyme precursor; apoenzyme; fluorescent substance; pigment; chemiluminescent compound; luminescent substance; coloring substance; magnetic substance; or a metal particle such as gold colloid; a radioactive substance such as ¹²⁵I, ¹³¹I, ³²P, ³H, ³⁵S, or ¹⁴C; a phosphorylated phenol derivative such as a nitrophenyl phosphate, luciferin derivative, or dioxetane derivative; or the like. The enzyme may be a dehydrogenase; an oxidoreductase such as a reductase or oxidase; a transferase that catalyzes the transfer of functional groups, such as an amino; carboxyl, methyl, acyl, or phosphate group; a hydrolase that may hydrolyzes a bond such as ester, glycoside, ether, or peptide bond; a lyases; an isomerase; or a ligase. The enzyme may also be conjugated to another enzyme.

The enzyme may be detected by enzymatic cycling. For example, when the detectable label is an alkaline phosphatase, a measurements may be made by observing the fluorescence or luminescence generated from a suitable substrate, such as an umbelliferone derivative. The umbelliferone derivative may comprise 4-methyl-umbellipheryl phosphate.

The fluorescent or chemiluminescent label may be a fluorescein isothiocyanate; a rhodamine derivative such as rhodamine β isothiocyanate or tetramethyl rhodamine isothiocyanate; a dancyl chloride (5-(dimethylamino)-1-naphtalenesulfonyl chloride); a dancyl fluoride; a fluorescamine (4-phenylspiro[furan-2(3H); 1ÿ-(3ÿH)-isobenzofuran]-3;3ÿ-dione); a phycobiliprotein such as a phycocyanine or physoerythrin; an acridinium salt; a luminol compound such as lumiferin, luciferase, or aequorin; imidazoles; an oxalic acid ester; a chelate compound of rare earth elements such as europium (Eu), terbium (Tb) or samarium (Sm); or a coumarin derivative such as 7-amino-4-methylcoumarin.

The label may also be a hapten, such as adamantine, fluoroscein isothiocyanate, or carbazole. The hapten may allow the formation of an aggregate when contacted with a multi-valent antibody or (strep)avidin containing moiety. The hapten may also allow easy attachment of a molecule to which it is attached to a solid support.

The label may be detected by quantifying the level of a molecule attached to a detectable label, such as by use of electrodes; spectrophotometric measurement of color, light, or absorbance; or visual inspection.

h. Peptide

A “peptide” or “polypeptide” as used herein may mean a linked sequence of amino acids and may be natural, synthetic, or a modification or combination of natural and synthetic.

i. Recombinant Polypeptide

A “recombinant polypeptide” or “recombinant protein” as used herein may mean at least a polypeptide of genomic, semisynthetic or synthetic origin which by virtue of its origin or manipulation is not associated with all or a portion of the polynucleotide with which it is associated in nature or in the form of a library, or is linked to a polynucleotide other than that to which it is linked in nature. The recombinant polypeptide may not necessarily be translated from a designated nucleic acid sequence of HBV. The recombinant polypeptide may also be generated in any manner, including chemical synthesis or expression of a recombinant expression system, or isolated from HBV.

j. Solid Support

“Solid support,” “solid phase,” or “solid substrate” as used herein may be the walls of wells of a reaction tray, test tubes, polystyrene beads, magnetic beads, nitrocellulose strips, membranes, microparticles such as latex particles, and others. The solid support is not critical and can be selected by one skilled in the art. Thus, latex particles, microparticles, magnetic or non-magnetic beads, membranes, plastic tubes, walls of microtiter wells, glass or silicon chips and sheep red blood cells are all suitable examples. Suitable methods for immobilizing peptides on solid supports include ionic, hydrophobic, covalent interactions and the like. The solid support may also be any material which is insoluble, or may be made insoluble by a subsequent reaction. The solid support may be chosen for its intrinsic ability to attract and immobilize the capture reagent. Alternatively, the solid support may retain an additional receptor which has the ability to attract and immobilize the capture reagent. The additional receptor may include a charged substance that is oppositely charged with respect to the capture reagent itself or to a charged substance conjugated to the capture reagent As yet another alternative, the receptor molecule may be any specific binding member which is immobilized upon (attached to) the solid support and which has the ability to immobilize the capture reagent through a specific binding reaction. The receptor molecule enables the indirect binding of the capture reagent to a solid support material before the performance of the assay or during the performance of the assay. The solid support thus may be a plastic, derivatized plastic, magnetic or non-magnetic metal, glass or silicon surface of a test tube, microtiter well, sheet, bead, microparticle, chip, and other configurations known to those of ordinary skill in the art.

It is contemplated and within the scope of the invention that the solid support also may comprise any suitable porous material with sufficient porosity to allow access by detection antibodies and a suitable surface affinity to bind antigens. Microporous structures are generally preferred, but materials with gel structure in the hydrated state may be used as well. Such useful solid supports include: natural polymeric carbohydrates and their synthetically modified, cross-linked or substituted derivatives, such as agar, agarose, cross-linked alginic acid, substituted and cross-linked guar gums, cellulose esters, especially with nitric acid and carboxylic acids, mixed cellulose esters, and cellulose ethers; natural polymers containing nitrogen, such as proteins and derivatives, including cross-linked or modified gelatins; natural hydrocarbon polymers, such as latex and rubber; synthetic polymers which may be prepared with suitably porous structures, such as vinyl polymers, including polyethylene, polypropylene, polystyrene, polyvinylchloride, polyvinylacetate and its partially hydrolyzed derivatives, polyacrylamides, polymethacrylates, copolymers and terpolymers of the above polycondensates, such as polyesters, polyamides, and other polymers, such as polyurethanes or polyepoxides; porous inorganic materials such as sulfates or carbonates of alkaline earth metals and magnesium, including barium sulfate, calcium sulfate, calcium carbonate, silicates of alkali and alkaine earth metals, aluminum and magnesium; and aluminum or silicon oxides or hydrates, such as clays, alumina, talc, kaolin, zeolite, silica gel, or glass (these materials may be used as filters with the above polymeric materials); and mixtures or copolymers of the above classes, such as graft copolymers obtained by initializing polymerization of synthetic polymers on a pre-existing natural polymer. All of these materials may be used in suitable shapes, such as films, sheets, or plates, or they may be coated onto or bonded or laminated to appropriate inert carriers, such as paper, glass, plastic films, or fabrics.

The porous structure of nitrocellulose has excellent absorption and adsorption qualities for a wide variety of reagents including monoclonal antibodies. Nylon also possesses similar characteristics and also is suitable. It is contemplated that such porous solid supports described hereinabove are preferably in the form of sheets of thickness from about 0.01 to 0.5 mm, preferably about 0.1 mm. The pore size may vary within wide limits, and is preferably from about 0.025 to 15 microns, especially from about 0.15 to 15 microns. The surfaces of such supports may be activated by chemical processes which cause covalent linkage of the antigen or antibody to the support. The irreversible binding of the antigen or antibody is obtained, however, in general, by adsorption on the porous material by poorly understood hydrophobic forces. Suitable solid supports also are described in U.S. patent application Ser. No. 227,272, which is incorporated herein by reference.

k. Specific Binding Member

“Specific binding member” as used herein may mean a member of a specific binding pair. The specific binding pair may be two different molecules where one of the molecules through chemical or physical means specifically binds to the second molecule. The specific binding member may be immunoreactive, and may be an antibody, an antigen, or an antibody/antigen complex that is capable of binding to a particular polypeptide.

l. Substantially Identical

“Substantially identical,” as used herein may mean that a first and second sequence are 50%-99% identical over a region of 8-100 or more residues.

m. Variant

“Variant” as used herein with respect to a polypeptide may mean (i) a portion of a referenced polypeptide which may be 8-100 or more amino acids; or (ii) a polypeptide that is substantially identical to a referenced polypeptide. A variant may also be a differentially processed polypeptide, such as by proteolysis, phosphorylation, or other post-translational modification.

### 2. TARGET

Provided herein is a method for detecting a target. The target may be any detectable molecule, such as a protein, nucleic acid, or nucleoprotein. The target may be capable of forming a complex with another molecule, which may decrease the sensitivity or detection of the target. It may be desirable to increase the sensitivity or detection of the target by displacing the target from a complex. The target may be of a virus, bacterium, parasite, fungus, plant, animal, or any other organism, or a cancer or cytokine. The presence of the target may be indicative of the organism, cancer, or cytokine. For example, the target may be indicative of a virus or viral infection.

Representative examples of viruses include a double-stranded DNA virus such as Adenovirus, Herpesvirus, or Poxvirus; a single-stranded (+)sense DNA virus such as a Parvovirus; a double-stranded RNA virus such as a Reovirus; a single-stranded (+)sense RNA virus such as a Picornavirus or Togavirus; a single-stranded (−)sense RNA virus such as an Orthomyxovirus or Rhabdovirus; a single-stranded (+)sense RNA virus with DNA intermediate in life-cycle, such as retroviruses; or a double-stranded DNA with RNA intermediate such as a Hepadnavirus. The virus may also be Human papilloma virus, HSV1, HSV2, RSV, EBV, Influenza A, Vaccinia virus, hepatitis B virus, hepatitis C virus, or human immunodeficiency virus. The HIV may be an isolate of HIV-1 group O, HIV-1 group M, and HIV-2.

Representative examples of bacteria include Bacillus anthracis, Brucella abortus, Cyanobacteria, Escherichia coli, Clostridium difficile, Lactobacillus bulgaricus, Mycobacterium tuberculosis, Pseudomonas aeruginosa, Mycoba, Rhizobia, Staphylococcus, Streptococcus species such as Streptococcus pneumoniae, Streptomyces griseus, and Thermus aquaticus. The S. pneumoniae targe may be a species-specific C-polysaccharide, a type-specific capsular polysaccharide, or pneumolysin. The P. aerugionosa target may be a prepared P. aeruginosa extract.

Representative examples of fungi include Cryptococci such as C. neoformans and Paracoccidioides species such as P. brasiliensis. The C. neoformans target may be a soluble capsular polysaccharide (CPS) antigen. The P. brasiliensis target may be an immunodominant antigen or gp43.

Representative examples of parasites include Trypanosoma cruzi and Plasmodium species such as Plasmodium falciparum, P. vivax, P. malariae, and P. ovale. The P. falciparum target may be a 175 kDa erythrocyte binding protein, EBA-175, a histidine-rich protein (PfHRP-2), a merozoite surface protein I (MSP1), or MSP2. The T. cruzi target may be a T. cruzi circulating antigen (cAg), an antigen such as Gp90, Gp 60/50, LPPG, FP10, FP6, FP3, or TcF, or a target as disclosed in U.S. Pat. Nos. 5,645,838, 5,623,058. 5,583,204, or 5,550,027, the contents of which are disclosed herein by reference.

The cancer target may be carcinoembryonic antigen (CEA), cancer antigen 15-3 (CA 15-3), or DF3 defined antigen. The cytokine may be IL-8.

a. Protein

(1) Hepatitis B Virus Protein

The protein may be a HBV protein or a variant thereof. The HBV protein may be a HBV core antigen (HBcAg), a HBV core-related antigen (HBcrAg), or a HBV e antigen (HBeAg), as disclosed in Kimura T, et al, J Clin Microbiol 2002 February; 40(2):439-45, the contents of which are incorporated herein by reference. The HBV protein may also be a HBV surface antigen protein (HBsAg), which may be capable of forming part of an HBV envelope. The HBsAg may be exposed on the surface of an HBV particle. The HBsAg may comprise an epitope, which may be antigenic or a target of immune surveillance. The epitope may be a mutant epitope. The HBsAg may also be glycosylated.

(a) Middle Hepatitis B Surface Antigen Protein (M-HBsAg)

The HBsAg may be a middle HBsAg (M-HBsAg). M-HBsAg may comprise a first portion and a second portion, and may have an overall length of about 281 amino acids. The first portion may comprise a preS2 region, and may be the first 55 amino acids of M-HBsAg. The second portion may be 226 amino acids in length and may comprise the sequence of S-HBsAg. M-HBsAg may comprise a sequence as set forth in Table 1 or a variant thereof. The first portion of M-HBsAg may also comprise an epitope. The epitope may be capable of being bound by an antibody. The antibody may be an anti-M-HBsAg-specific antibody.

TABLE 1

| | |
|---|---|
| SEQ | |
| ID | |
| NO | Middle HBV Surface Antigen Protein |

| | | |
|---|---|---|
| 1 | 1 | mqwnsttfhq tlqdprvral yfpaggsssg tvspaqntvs |
| | | aissilsktg dpvpnmenia |
| | 61 | sqllgpllvl qagfflltki ltipqsldsw wtslnflggt |
| | | pvclgqnsqs qisshsptcc |
| | 121 | ppicpgyrwm clrrfiiflc illlclifll vlldyqgmlp |
| | | vcplipgsst tstgpcktct |
| | 181 | tpaqgtsmfp sccctkptdg nctcipipss wafakyiwew |
| | | asvrfswlsl lvpfvqwfvg |
| | 241 | lsptvwlsvi wmmwywgpsl ynilspfmpl lpiffclwvy |
| | | i |

(b) Small Hepatitis B Surface Antigen Protein (S-HBsAg)

The HBsAg may also be a small HBsAg (S-HBsAg). S-HBsAg may be about 226 amino acids in length, and may comprise a S region. The S-HBsAg may be a wild-type S-HBsAg. The S-HBsAg may comprise a sequence as set forth in Table 2, or a variant thereof. S-HBsAg may also comprise an epitope, which may be part of an “a” determinant as disclosed in U.S. Pat. No. 5,925,512 or 7,141,242, the contents of which are incorporated herein by reference. Amino acids 100-160 of S-HBsAg may also comprise an epitope.

TABLE 2

| | |
|---|---|
| SEQ | |
| ID | |
| NO | Small HBV Surface Antigen Protein |

| | | |
|---|---|---|
| 2 | 1 | meniasgllg pllvlqagff lltkiltipq sldswwtsln |
| | | flggtpvclg qnsqsqissh |
| | 61 | sptccppicp gyrwmclrrf iiflcilllc liflivildy |
| | | qgmlpvcpli pgssttstqp |
| | 121 | cktcttpaqg tsmfpsccct kptdgnctci pipsswafak |
| | | ylwewasvrf swlsllvpfv |
| | 181 | qwfvqlsptv wlsviwmmwy wgpslynils pfmpllpiff |
| | | clwvyi |

(2) Hepatitis C Virus Protein

The protein may be a HCV protein or a variant thereof. The HCV protein may comprise a polyprotein as set forth in GenBank Accession No. P27958. The HCV protein may comprise the core or nucleocapsid protein, which may comprise the first 191 amino acids of the polyprotein. The core protein may comprise an antigen, which may be a core antigen, NS3, NS4, or NS5.

(3) Human Immunodeficiency Virus Protein

The protein may be a HIV protein or a variant thereof. The HIV protein may be a gag-pol polyprotein or gag-pol polyprotein precursor (Pr180gag-pol). The HIV protein may also be a pol precursor or a gag precursor (Pr55gag). The HIV protein may also comprise a p17 (myristilated gag protein), p24 (major structural protein), p7 (nucleic acid binding protein), or p9 (proline-rich protein) Pr55gag protein cleavage product. The HIV protein may also comprise a gp 160 envelope polyprotein precursor, or a gp 160 cleavage product such as gp120 (envelope glycoprotein) or gp41 (transmembrane protein).

b. Antibody

The target may also be an antibody, which may be capable of binding to a protein, nucleic acid, or nucleoprotein, which may be viral. The antibody may be capable of binding an antigen of the protein or nucleoprotein. The antibody may be generated by an immune reaction of the subject to the protein, nucleic acid, or nucleoprotein.

### 3. METHOD FOR DETECTING A TARGET

Provided herein is a method for detecting a target in a sample. The target may be bound to a first binding partner, which may reduce the sensitivity of detecting the target. Diplacing the target/first binding partner complex may free the target and facilitate detection of the target. For example, the first binding partner may occlude an antigen of a target protein. By displacing the target protein from the first binding partner, the antigen may be more readily detectable. Upon displacing the target/first binding partner complex, the first binding partner may be removed from the sample. The sample may be contacted with a second binding partner, which may be capable of binding the target. The target/second binding partner complex may be detected, and the presence of this complex in the sample may be indicative of the presence of the target. The amount of target/second binding partner complex may also be indicative of the amount of target in the sample. The displacement and detection steps may be performed in at least duplicate. The displacement, removal, and detection steps may also be performed in at least duplicate.

a. Displacing

(1) First binding partner

The target may be bound to a first binding partner, which may be a protein, nucleic acid, nucleoprotein, or other such molecule. The first binding partner protein may be an antibody, which may be capable of binding to the target or an antigen of the target. The antibody may be the result of an immune response of the subject to the target.

The first binding partner protein may also be an antigen, which may be viral. The antigen may be capable of binding to an antibody, which may be the result of an immune response of a subject to the first binding partner antigen.

(2) Displacing Agent

The target/first binding partner complex may be displaced by using a displacing agent, which may be a low pH. The pH may be 2-6. This pH may be achieved by using a solution with a pH of 1-2. The solution may be a glycine reagent with a glycine concentration of 50 mM to 1M. Low pH may also be achieved by using hydrochloric acid or acetic acid. Following disruption by the displacement agent, the sample may be neutralized using a neutralizing agent, which may be 500 mM to 2M Tris, and may have a pH of 7 to 12. The displacing agent may also be high pH, which may be a pH of 10 to 13. High pH may be achieved by using borate, phosphate acid, diethylamine, or triethylamine.

The displacing agent may also be heat, and may be a temperature of 60° C. to 110° C. The displacement agent may also be an agent capable of reducing disulfide bonds, such as beta mercaptoethanol, dithiothreitol, glutathione, cysteine, or Tris(2-carboxyethyl)phosphine hydrochloride. The displacement agent may also be a concentrated salt solution, which may have a salt concentration of 1 to 5M, which may be achieved by using MgCl₂ or LiCl. The displacing agent may be urea, a detergent such as SDS, or a chaotropic agent such as sodium thiocyanate.

Upon displacing the target/first binding partner complex, the first binding partner may be removed, which may be accomplished by using a first binding partner binding reagent. The first binding partner binding reagent may an antibody-binding reagent, which may be an anti-human Ig. The first binding partner binding reagent may also be an antigen capable of binding to the first binding partner. The antibody binding reagent may also be Protein A/G, Protein G, or Protein A. The antibody binding reagent may be attached to a solid support.

b. Contacting

Upon displacing the first binding partner from the target in the sample, the sample may be contacted with a second binding partner, which may be capable of binding to the target. The second binding partner may be more readily capable of binding to the target when the target has been displaced from the first binding partner. The contacting may occur for a time and under conditions sufficient for the formation of a target/second binding protein complex. The contacting step may be performed concurrently with displacing the target/first binding partner complex.

(1) Second Binding Partner

The second binding partner may comprise a label, and may be part of an indicator reagent. The second binding partner may also be attached to a solid support.

The second binding partner may comprise an antibody such as an anti-antibody, which may be capable of binding to a virus-reactive antibody. The virus-reactive antibody may be capable of binding to a viral protein or antigen.

The second binding partner may also comprise an antigen, which may be capable of binding to the target. The target may be a virus-reactive antibody. The second binding partner antigen may be viral, recombinant, or synthetic.

(a) Hepatitis B Virus Second Binding Partner

The second binding partner may be capable of binding an antigen of the HBV protein. The second binding partner may be a 50-80, 116-34, H166, H57, H40, H53, or H35 monoclonal anti-HBs antibody, or a similar antibody.

(b) Hepatitis C Virus Second Binding Partner

The second binding partner may a HCV second binding partner, and may be capable of binding to an antigen of the HCV protein. The second binding partner may be a 13-959-270, 14-1269-281, 14-1287-252, 14-153-234, 14-153-462, 14-1705-225, 14-1708-269, 14-1708-403, 14-178-125, 14-188-104, 14-283-112, 14-635-225, 14-726-217, 14-886-216, 14-947-104, 14-945-218, 13-975-157, 14-1350-210, 107-35-54, 110-81-17, C11-3, C11-7, C11-10, C11-14, or C11-15 anti-HCV protein monoclonal antibody, or a similar antibody.

The HCV second binding partner may be an antigen capable of binding to a HCV-reactive antibody. The antigen may be core antigen, NS3, NS4, NS5, or a variant thereof. The antigen may also be p380-JH1, p380.LG, p380-J, or p408J, as described in U.S. Pat. No. 6,596,476, the contents of which are incorporated herein by reference.

(c) HIV Protein Second Binding Partner

The second binding partner may be capable of binding to the HIV target, and may be a 120A-270, 115B-151, 117-289, 108-394, 115B-303, or 103-350 monoclonal antibody, or other antibody as disclosed in U.S. Pat. Pub. No. 2002/0606636 A1, the contents of which are incorporated herein by reference.

c. Detecting

Upon contacting the target with the second binding partner, the target/second binding partner complex may be detected. This may be accomplished by contacting the sample with the second binding partner for a time and under conditions sufficient to form a target/second binding partner complex, and measuring the level of detectable signal from the target/second binding partner complex. The level of signal detected in the sample may indicate the presence of the target, and may also indicate the amount of the target. The level of the signal may be proportional to the amount of target in the sample. The level of the signal may be compared to a control. A difference in the level of signal detected from the target/second binding protein complex in the sample compared to a control may be indicative of the presence or amount of the target. The detection step may be performed together with the displacing and contacting steps, such as in the first incubation step of a one-step capture/detection procedure.

If the target is a viral antigen that is bound to a anti-viral antigen antibody, then upon displacement of the anti-viral antigen antibody from the viral antigen in a sample, the target may be detected by contacting the sample with an indicator reagent comprising an anti-viral antigen antibody for a time and under conditions sufficient to form a detection target viral antigen/detection anti-viral antigen antibody complex. The level of the target may be determined by detecting the measurable signal generated by the label.

If the target is an anti-viral antibody that is bound to a viral antigen, then upon displacement of the viral antigen from the anti-viral antigen antibody in the sample, the target may be detected by contacting the sample with an indicator reagent comprising an anti-anti-viral antibody for a time and under conditions sufficient to form an anti-viral antibody/detection anti-anti-viral antibody complex. The level of the target may be determined by measuring the detectable signal generated by the label.

If the target is an anti-viral antibody, then upon displacement of the viral antigen from the anti-viral antibody in the sample, the target may also be detected by contacting the sample with an indicator reagent comprising a viral antigen for a time and under conditions sufficient to form an anti-viral antibody/detection viral antigen complex. The level of the target may be determined by measuring the detectable signal generated by the label.

A non-solid phase diagnostic assay may be used in the method. These assays are well-known to those of ordinary skill in the art and are considered to be within the scope of the present invention. Examples of such assays include those described in U.S. Pat. No. 5,925,512 or 7,141,242, the contents of which are incorporated herein by reference.

The label may be detected using a detection system, which may comprise a solid support. The solid support may be adapted to be used by a semi-automated or fully automated immunoanalyzer. The detection system may deliver the sample and reagents (which may comprise an antigen, an antibody, a label, a buffer, or the like) to a reaction vessel, perform incubations, and optionally wash an unbound labeled polypeptide from a bound labeled polypeptide. The detection system may be automated without user intervention once the sample and reagents are inserted into the system. The automated detection system may be distinguished from a manual or less-automated system by the ability of the system to perform at least 8, 16, 64 or 128 assays in a 48-hour period without user intervention. The system may also be able to calculate the concentration or quantity of a polypeptide in the sample automatically, without the need for human calculation or input.

The detection system may also comprise a cartridge format or test strip assay. The detection system may provide unit-dose loadable assay reagents into a disposable instrument, and the unit-dose may contain all the reagents necessary to assay to detect the polypeptide. The disposable instrument may comprise a plastic housing, which may comprise a disposable membrane-like structure of nylon, nitrocellulose, or other suitable material. The sample may be preprocessed or loaded directly onto a loading zone of the disposable instrument. The sample may then optionally flow across the membrane-like structure through a plurality of zones contained on the membrane. The membrane-like structure may further comprise a detergent or lateral flow-aid. The membrane-like structure may also contain an absorbant to collect excess fluid and/or encourage lateral flow across the membrane. The detection system may comprise a multi-pack system in which each pack may comprise sufficient reagents to perform 1, 2, 4, 8, 10, or 12 assays.

The detection system may also comprise a microfluidic device designed to analyze the sample in the microliter range (e.g., less than 4 μL, 12 μL, 50 μL, 250 μL). The microfluidic device may comprise a flow aids, propulsion device (which may comprise an expansion gel, wax, or gas), nanovalving, or the like to assist the transportation of the sample or assay reagents or both through the microfluidic device.

Of course, it goes without saying that any of the exemplary formats herein, and any assay or kit according to the invention can be adapted or optimized for use in automated and semi-automated systems (including those in which there is a solid phase comprising a microparticle), as described, e.g., in U.S. Pat. Nos. 5,089,424 and 5,006,309, the contents of which are incorporated herein by reference, and as, e.g., commercially marketed by Abbott Laboratories (Abbott Park, Ill.) including but not limited to Abbott's ARCHITECT®, AxSYM, IMX, PRISM, and Quantum II platforms, as well as other platforms.

Additionally, the assays and kits described herein optionally can be adapted or optimized for point of care assay systems, including Abbott's Point of Care (i-STAT™) electrochemical immunoassay system. Immunosensors and methods of manufacturing and operating them in single-use test devices are described, for example in U.S. Pat. No. 5,063,081 and published U.S. Patent Application Publication Nos. 20030170881, 20040018577, 20050054078, and 20060160164, the contents of which are incorporated herein by reference.

### 4. SAMPLE

The sample comprising the target may be isolated from a patient. The sample may be a biological tissue or fluid isolated from an animal, such as a human. The sample may also be a section of tissue such as a biopsy or autopsy sample, a frozen section taken for histologic purposes, blood, plasma, serum, sputum, stool, tears, mucus, hair, or skin. The sample may also be an explant, or primary or transformed cell culture derived from an animal or patient tissue. The sample may be provided by removing a sample of cells from an animal, but may also be accomplished by using previously isolated cells (e.g., isolated by another person, at another time, and/or for another purpose). An archival tissue, such as that having treatment or outcome history, may also be used. The sample may also be blood, a blood fraction, effusion, ascitic fluid, saliva, cerebrospinal fluid, cervical secretion, vaginal secretion, endometrial secretion, gastrointestinal secretion, bronchial secretion, sputum, cell line, tissue sample, or secretion from the breast.

The sample may have a volume equal to or greater than 20 μL. The sample may also have a volume equal to or greater than 250 μL.

### 5. PATIENT

The patient may be infected with a virus, which may be hepatitis B virus, hepatitis C virus, or human immunodeficiency virus. The infection may be an occult infection, such as a HBV occult infection.

### 6. KIT

Provided herein is a kit, which may be used for detecting the target. The kit may comprise the displacing agent, the second binding partner, and may comprise an indicator reagent comprising the second binding partner. The kit may also comprise the first binding partner binding reagent. The kit may also comprise a solid support suitable for binding proteins from a sample. The kit may also comprise a composition comprising the target at a known concentration for use as a positive control.

The kit may also comprise an additional reagent such as a buffer or salt, which may be required for promoting or preventing protein-protein interactions, or removing unbound proteins from a solid support. The kit may further comprise an agent capable of inducing a label on an indicator reagent to generate a detectable signal. The kit may also comprise an agent capable of stopping a label from generating a signal.

The kit may also comprise one or more containers, such as vials or bottles, with each container containing a separate reagent. The kit may further comprise written instructions, which may describe how to perform or interpret an assay described herein.

The present invention has multiple aspects, illustrated by the following non-limiting examples.

### EXAMPLE 1

### Detecting Hepatitis B Surface Antigen Upon Displacing HBsAg/Anti-HBsAg Antibody Complexes Improves Detection of Samples with Immune Complexes

This example demonstrates that detecting a target protein upon disrupting target/antibody immune complexes improves the sensitivity of detecting the target in control immune complex samples. Immune complex samples were prepared by combining a sample containing anti-HBs with a solution of HBsAg (subtype ad) diluted to 150 pg/ml in normal human plasma. The amount of antibody in the immune complex samples was varied by diluting the anti-HBs sample by a factor of 10, 25, 50, 100, or 200 in the 150 pg/ml HBsAg solution. The mixture was incubated for at least 1 h at room temperature and then used in a prototype Abbott PRISM HBsAg assay (Abbott Laboratories, Abbott Park, Ill.) or stored at 4° C. The samples will serve as immune complex control samples to determine whether the signal is increased relative to treatment with low pH to dissociate immune complexes.

Prior to detecting HBsAg in the anti-HBs/HBsAg complex samples, samples were spun at 14,000 rpm for 15 min in an Eppendorf 5415C Microfuge (New York, N.Y.). A number of conditions for treating sample with low pH followed by neutralization were attempted during the optimization and these are indicated in Table 3. To lower the pH of the sample, the glycine reagents listed are 250 mM glycine, pH 1.5, 250 mM glycine, 0.3% BSA, pH 1.5, and 50 mM glycine, 0.3% BSA, pH 2.0. The control reagent for untreated samples was PBS, 0.3% BSA. The neutralizing agents listed are 1M Tris, pH 9.0, 1M Tris, pH 7.4, and 2M Tris, pH 11.5.

For treated samples, the final pH of each sample ranged from 2 to 6, as measured using colorpHast strips (Merck KGaA, Darmstadt, Germany) 0 to 14. For the untreated samples, PBS, 0.3% BSA was added in place of the glycine reagent. Samples were then incubated at 37° C. for 30 to 60 min with rotation on a Dynamic Incubator (DI; Abbott Laboratories, Abbott Park, Ill.). Next, treated samples were neutralized with Tris to a pH of 6-9 as measured using colorpHast strips 5 to 10. For untreated samples, PBS or Tris solution was added in the neutralization step.

TABLE 3
Treatment Conditions

| | Conditions | | | | | | | | | | | | | | |
|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 |
| μL Gly, pH 1.5 | 250 | | | | 500 | 700 | | | | | | 1000 | | | |
| μL GlyBSA, pH 1.5 | | | | | | | | | 250 | 250 | | | | 1000 | |
| μL GlyBSA, pH 2.0 | | 500 | 700 | 700 | | | 500 | 500 | | | 250 | | 1000 | | 250 |
| μL Water | 250 | | | | | | | | 250 | 250 | 250 | | | | 750 |
| μL Tween 20, 10% | | | | | | | | 7.5 | | 10 | | | | 18 | |
| μL Sample | 250 | 250 | 250 | 250 | 250 | 250 | 250 | 250 | 250 | 250 | 250 | 500 | 500 | 500 | 250 |
| Minutes (37 C., DI) | 60 | 30 | 60 | 30 | 60 | 60 | 60 | 30 | 60 | 60 | 60 | 60 | 60 | 60 | 60 |
| μL 1M Tris, pH 9 | | | 50 | 50 | | 50 | | | | | 20 | 300 | | 300 | 20 |
| μL 1M Tris, pH 7.4 | 250 | | | | | | | | 250 | 250 | | | | | |
| μL 2M Tris, pH 11.5 | | 10 | | | 10 | | 10 | 10 | | | | | 25 | | |

Following treatment for immune complex dissociation, samples were then spun at 14,000 rpm for 15 min in an Eppendorf Microfuge. As in the PRISM HBsAg assay, 50 μL of microparticles were then incubated with the samples for 18 min at 37° C. (DI without rotation). The microparticles were coated with two anti-HBs monoclonal antibodies, H166 and 116-34. Following incubation, samples were spun in an Eppendorf Microfuge at 10,000 rpm for 5 min. Supernatants were removed and 1 mL of PRISM HBsAg Transfer Wash (PRISM HBsAg 3A47C) was added to the tubes. Tubes were spun in an Eppendorf Microfuge at 10,000 rpm for 5 min. Supernatants were removed and 100 μL of PBS was added to the tubes. Tubes were then spun briefly and the microparticle pellets were resuspended 5 times with a pipette.

Reacted microparticles (100 μL) were added to the sample well of an Abbott PRISM Reaction Tray (Abbott Laboratories, Abbott Park, Ill.). Nothing was dispensed into the tray by the instrument until the Transfer Wash station. At this juncture, the tray was subjected to Abbott PRISM immunoanalysis per the manufacturer's instructions (Abbott Laboratories, Abbott Park, Ill.). The conjugate was comprised of acridinium-conjugated goat anti-HBs polyclonal at 150 ng/mL and acridinium-conjugated anti-HBs H35 at 130 ng/mL. The instrument sequences used for the commercial PRISM HBsAg assay were utilized with the exception of the Conjugate Wash protocol. This step was altered to include one wash of 300 μL borate buffered saline (BBS), followed by two washes of 100 μL BBS. Signal or relative light units (rlus) were measured on the PRISM instrument.

Of the assay conditions indicated in Table 3, conditions 7, 9, 13 and 15 resulted in the lowest background counts for HBV negative plasma samples and minimal issues with microparticle clumping. From Table 3, conditions 7, 9, 13, and 15 were used to test immune complex samples with the anti-HBs sample diluted by a factor of 50 (DF50) in the 150 pg/ml HBsAg sample. The results of testing these samples are found in Table 4. The ratio of treated sample rlu to untreated sample rlu is shown for comparison. Condition 9 resulted in the largest increase in rlu values for treated as compared to untreated immune complex control sample.

TABLE 4
Results of Varying Treatment Conditions to Dissociate
Immune Complexes

| | Conditions | | | |
|---|---|---|---|---|
| | 7 | 9 | 13 | 15 |
| Sample | Treat:Untreat | Treat:Untreat | Treat:Untreat | Treat:Untreat |
| HBsAg at | 0.9 | 1.0 | 1.0 | 1.2 |
| 150 pg/ml | | | | |
| Immune | 1.1 | 2.2 | 1.2 | 1.7 |
| Complex | | | | |
| Sample DF50 | | | | |
| Rep 1 | | | | |
| Immune | 0.8 | 1.9 | 1.4 | 1.7 |
| Complex | | | | |
| Sample DF50 | | | | |
| Rep 2 | | | | |

The detectable signals measured from negative samples were used to calculate a cut-off (CO) value for the treated (i.e., with low pH) and untreated (i.e., with PBS) samples. The experiment was performed according to the general detection method indicated above and assay condition 9 (Table 3). The CO values were the mean of the negative samples, plus 5 standard deviations. For the untreated samples, the CO value was 373 (mean=187, SD=37.1), and for the treated samples, the CO value was 358 (mean=218, SD=28.1).

Immune complex control samples with varying amounts of anti-HBs were treated with low pH and the values compared to samples that were not treated with low pH (Table 5). The experiment was performed according to the general detection method indicated above and assay condition 9 (Table 3). For the treated samples in which anti-HBs was diluted by a factor of 100 or 200, the signal to cut-off (S/CO) values were greater than 1. The ratio of rlu or S/CO of treated:untreated sample was 1.5 or greater, indicating that displacing HBsAg from immune complexes with anti-HBs prior to detecting HBsAg improves signal. This demonstrates that HBsAg assays with steps included to dissociate anti-HBs from HBsAg will result in improved detection of samples with immune complexes. This should lead to an improved performance of the HBsAg assay when testing occult samples.

TABLE 5
Results of Treating Immune Complex Control Samples
to Dissociate Immune Complexes

| Sample | Treat rlu | Untreat rlu | Treat:Untreat |
|---|---|---|---|
| Immune Complex Sample DF10 | 239 | 161 | 1.5 |
| Immune Complex Sample DF25 | 315 | 154 | 2.0 |
| Immune Complex Sample DF50 | 326 | 172 | 1.9 |
| Immune Complex Sample DF100 | 430 | 177 | 2.4 |
| Immune Complex Sample DF200 | 548 | 281 | 2.0 |

### EXAMPLE 2

### Increasing Sample Volumes and Detecting Hepatitis B Surface Antigen Upon Displacing HBsAg/Anti-HBsAg Antibody Complexes Improves Sensitivity and Detection of Samples with Potential Immune Complexes

This example demonstrates that detecting a target protein upon disrupting target/immune complexes increases the sensitivity of detecting the target and compares these results to previous testing. The general detection method of Example 1 was used to measure the level in treated and untreated samples. The treatment method to dissociate immune complexes is found in Table 6 and was used for all testing in this example. The sample volume used in the assay with immune complex dissociation was 250 μL. In the analysis of the immune dissociation test results, the level of signal measured from treated (displaced) and untreated (no displacement) samples containing HBsAg were compared to each other and also to their respective CO values.

The analytical sensitivity values of the assay using 250 μL sample volume and immune complex dissociation steps was determined by testing members of the Abbott HBsAg Sensitivity panel. This was conducted to gain information on the impact of using 250 μL of sample and the impact of the low pH treatment on the assay performance. In the final HBsAg assay configuration, all samples would be treated to dissociate immune complexes, so maintaining analytical sensitivity is important. For the ad subtype, the analytical sensitivity of the untreated sample format was 0.042 ng/mL, and the treated samples had a sensitivity of 0.018 ng/mL. Accordingly, measuring HBsAg levels upon displacement of HBsAg/anti-HBs complexes did not impair the analytical sensitivity in this experiment and in fact there was increased sensitivity of HBsAg detection in the treated samples.

Samples numbered 401 and 408 were known to be positive for HBV. These samples were previously tested in the PRISM HBsAg Prototype 2 assay. In the PRISM HBsAg Prototype 2 assay, 1000 μL of the sample was used in the general method of Example 1, but without immune complex displacement. The analytical sensitivity of the PRISM HBsAg Prototype 2 assay is 0.008 to 0.009 ng/mL for subtypes ad/ay.

While anti-HBs-HBsAg immune complexes were not detectable in sample 408 using displacement (i.e., the treated:untreated ratio was less than 1), varying the conditions only by additionally increasing the sample volume from 250 μL to 1000 μL changed the S/CO values from untreated (non-displaced) values of 4.40 and 4.90 (Table 7) to a range of 7.94 to 10.82. Accordingly, increasing sample volume without immune complex displacement also improves the sensitivity of detecting HBsAg.

Sample 401 was negative for HBsAg in the standard commercial version of the PRISM HBsAg assay (Abbott PRISM HBsAg Assay, Abbott Laboratories, Abbott Park, Ill.). However, HBsAg was detectable from sample 401 when 1000 μL of the sample were used in the general method of Example 1, but without immune complex displacement (PRISM HBsAg Prototype 2). In the PRISM HBsAg Prototype 2 assay, the S/CO range for sample 401 was 0.80 to 1.54 and this sample was detected 3 out of 5 times. Accordingly, increasing sample volume without immune complex displacement improves the sensitivity of detecting HBsAg and also replicate testing improves detection.

Treatment of sample 401 to dissociate immune complexes increased S/CO values. In Table 7, the S/CO values and a comparison of Treated:Untreated S/CO values show the elevated signal after displacement as compared to the values without displacement. Accordingly, displacing target proteins from immune complexes together with increasing sample volume when detecting the target proteins also increases the detection of the target proteins.

TABLE 6
Assay Conditions Used to Test Samples with
Potential Immune Complexes

| Condition | |
|---|---|
| μL GlyBSA, pH 1.5 | 250 |
| μL Water | 250 |
| μL Sample | 250 |
| Minutes (37 C., DI) | 60 |
| μL 1M Tris, pH 7.4 | 250 |

TABLE 7
Results of Treating Samples with Potential Immune Complexes

| Sample | Treat S/CO | Untreat S/CO | Treat:Untreat S/CO |
|---|---|---|---|
| 401, Experiment 1 | 1.68 | 0.80 | 2.1 |
| 401, Experiment 2 | 1.82 | 0.75 | 2.4 |
| 408, Experiment 1 | 3.68 | 4.40 | 0.8 |
| 408, Experiment 2 | 4.07 | 4.90 | 0.8 |

### EXAMPLE 3

### Detecting Hepatitis B Surface Antigen Upon Displacing HBsAg/Anti-HBsAg Antibody Complexes Improves Detection of Samples with Immune Complexes in a Magnetic Microparticle Assay

This example demonstrates that detecting a target protein upon disrupting target/antibody immune complexes improves the detection of target in samples that contain immune complexes in an assay format that utilizes magnetic microparticles and a KingFisher (KF) instrument. The KingFisher is a microtiter plate sample processor that moves magnetic microparticles from well to well of 96 well plates. There are 12 magnets that are covered with a disposable cover or tip comb. It is possible to release the microparticles into wells by withdrawing the magnet from the comb and to pick-up microparticles by inserting the magnet into the comb. Prior to processing samples on the KF instrument, sample and reagents are added to the plate. An instrument sequence was selected and the plate was processed on the KF. A total of 12 samples could be tested with one microtiter plate. Sample and reagents were added to rows A through H. In the last step of the assay, the microparticles with bound sample and conjugate were added to a well containing ARCHITECT Pretrigger solution. The plate was moved to a microtiter plate reader, triggered, and the signal was measured.

Immune complex samples were prepared by combining a sample containing anti-HBs with a solution of HBsAg (subtype ad) diluted to 250 pg/mL in normal human plasma. This sample was referred to as HBsAg 250 pg/mL. The anti-HBs was diluted by a factor of 50 in the 250 pg/mL HBsAg solution. The mixture was incubated for at least 1 h at room temperature and then used in a HBsAg assay or stored at 4° C. This sample served as immune complex control (IC Sample) to determine whether the signal is increased relative to treatment with low pH to dissociate immune complexes.

Several conditions for treating sample with low pH followed by neutralization were attempted and these are indicated in Table 8. To lower the pH of the sample, a 1M glycine solution at varying concentrations was added. For the untreated samples, PBS was added in place of the glycine reagent. The neutralizing agent for the control containing PBS was 10.5M Tris, pH 7.4 and for the sample treated with glycine solution, it was 2M Tris, pH 11.5.

The reagents and volumes as indicated in Table 9 were added to a microtiter plate. Sample was added last and immediately after addition, the plate was incubated at 37° C. for 15 min with rotation on a Dynamic Incubator (DI; Abbott Laboratories, Abbott Park, Ill.). For treated samples, the final pH of each sample ranged from 2 to 4, as measured using colorphast strips (Merck KGaA, Darmstadt, Germany) 0 to 6 (Table 10). At the end of 15 minutes, the samples were neutralized with Tris. The pH of the sample after Tris addition was at a pH of 7 to 8 as measured using colorpHast strips 5 to 10.

TABLE 8
Treatment and Sample Volumes

| Code | A | B | C | D | E |
|---|---|---|---|---|---|
| ul 1M Glycine | 20 | 30 | 40 | 41.5 | 62.25 |
| ul Water | 40 | 30 | 20 | 0 | 0 |
| ul Sample | 100 | 100 | 100 | 125 | 125 |

15 Minutes at 37° C. (DI)

| | | | | | |
|---|---|---|---|---|---|
| ul 2 M Tris | 9 | 13 | 17 | 17 | 27 |

TABLE 9
Reagent Placement/Volumes in Microtiter Plate for
KF HBsAg Assay (Format 1)

| Well | Reagent | ul Volume |
|---|---|---|
| A | Sample/Treatment | See Table 8 |
| B | Microparticles | 50 |
| C | Architect WB | 150 |
| D | Conjugate | 50 |
| E | Conjugate Wash | 150 |
| | Buffer (CWB) | |
| F | CWB | 150 |
| G | CWB | 150 |
| H | Archtitect | 50 |
| | Pretrigger | |

TABLE 10
Measurement of Sample pH After Reagent Addition

| | Conditions | | | | |
|---|---|---|---|---|---|
| | A | B | C | D | E |
| mM Glycine | 125 | 183 | 250 | 250 | 375 |
| ul Sample | 100 | 100 | 100 | 125 | 125 |
| pH Sample | 3.5 | 3.0 | 2.5 | 2.5 | 2.0 |

Following treatment for immune complex dissociation, the microtiter plate with neutralized sample and reagent was placed in the KF instrument. The instrument sequence began by adding microparticles coated with anti-HBs monoclonal antibody, H166, from row B to row A, wells 1 to 12. The sample and microparticles were incubated for 18 minutes and then the magnetic microparticles were moved from row A to row C. After a wash step, the microparticles were moved from row C to row D. The microparticles were incubated with conjugate for 8 minutes and then followed by sequential wash steps in rows E, F and G. The conjugate comprised acridinium-conjugated goat anti-HBs polyclonal at 250 ng/mL and acridinium-conjugated anti-HBs H35 at 100 ng/mL. The conjugate wash buffer (CWB) comprised 0.5% dodecyltrimethylammonium bromide (Sigma D8638) and 1M sodium chloride in 50 mM MES at pH 6.3. After washing, the microparticles were moved from row G to H. The microparticles and ARCHITECT Pretrigger were incubated for 2 minutes and the microparticles are moved to well G. At the conclusion of the instrument sequence, the microtiter plate was moved to a microtiter plate reader where 150 μL of ARCHITECT Trigger was injected into designated wells, in this case row H, and read for 3 seconds.

In Table 11, the signal to noise (S/N) ratios for the IC Sample for all conditions tested are shown. The S/N value was calculated by dividing the sample rlu by the normal human plasma (NC) rlu. The highest S/N values for the IC Sample were obtained using condition D. A comparison of Treated:Untreated values shows the elevated signal after displacement as compared to the values without displacement. Using Condition D, the Treated:Untreated value for the IC Sample was 5.5 as compared to 1.0 obtained when testing the HBsAg 250 pg/ml sample, indicating that displacing HBsAg from immune complexes with anti-HBs prior to detecting HBsAg improved signal. This demonstrates that HBsAg assays with steps included to dissociate anti-HBs from HBsAg will result in improved detection of samples with immune complexes.

TABLE 11
Results of Varying Conditions to Dissociate Immune Complexes

| | Conditions | | | | |
|---|---|---|---|---|---|
| | A | B | C | D | E |
| Sample | TR S/N | TR S/N | TR S/N | TR S/N | TR S/N |
| HBsAg 250 pg/ml | 9.6 | 9.7 | 10.7 | 12.2 | 6.2 |
| IC Sample | 2.0 | 3.0 | 4.8 | 5.3 | 3.3 |
| | | | | TR:UN | TR:UN |
| HBsAg 250 pg/ml | | | | 1.0 | 1.0 |
| IC Sample | | | | 5.5 | 3.4 |

### EXAMPLE 4

### Detecting Hepatitis B Surface Antigen Upon Displacing HBsAg/Anti-HBsAg Antibody Complexes does not Impair Precision or Assay Sensitivity and Improves Detection of Samples with Potential Immune Complexes in a Magnetic Microparticle Assay

This example demonstrates that detecting a target protein upon disrupting target/immune complexes increases the sensitivity of detecting the target. Unless indicated, the general detection method of Example 3 and the same reagents as used in Example 3 were used to measure the level in treated and untreated samples. The treatment method to dissociate immune complexes is found in Table 12 and was used for all testing in this example. For untreated samples, the method discussed in Example 3 was followed. The sample volume used in the assay was increased from 125 to 250 μL. Increasing sample volume resulted in increased signal when testing the HBsAg 250 pg/mL sample.

TABLE 12
Assay Treatment Volumes and Incubation

| Condition | Per Well |
|---|---|
| μL 1M Glycine, pH 1.5 | 41.5 |
| μL Sample | 125 |
| Minutes (37 C., DI) | 15 |
| μL 2M Tris, pH 11.5 | 17 |

TABLE 13
Reagent Placement and Volumes in Microtiter Plate for KF HBsAg Assay
(Format 2)

| Well | Reagent | ul Volume |
|---|---|---|
| A | Sample/Treatment | See Table 12 |
| B | Sample/Treatment | See Table 12 |
| C | Microparticles | 50 |
| D | Architect WB | 150 |
| E | Conjugate | 50 |
| F | Conjugate Wash Buffer (CWB) | 150 |
| G | CWB | 150 |
| H | Archtitect Pretrigger | 50 |

To determine whether the treatment protocol has an effect on the precision of the KF HBsAg assay, the samples were tested in replicates of four using both the untreated and treated conditions. Following treatment for immune complex dissociation, the microtiter plate with neutralized sample and reagent was placed in the KF instrument. The placement of reagents in wells of the microtiter plate and the volumes added are found in Table 13. The instrument sequence began by adding microparticles from row C to row A, wells 1 to 12. The sample and microparticles were incubated for 9 minutes and then the magnetic microparticles were moved from row A to row B. The sample in row B and microparticles were incubated 9 minutes and then the magnetic microparticles were added to row D. After washing, the microparticles were moved from row D to row E. The microparticles were incubated with conjugate for 8 minutes followed by sequential wash steps in rows F and G. After washing, the microparticles were moved from row G to H. The microparticles and ARCHITECT Pretrigger were incubated for 2 minutes and the microparticles are moved to well G. At the conclusion of the instrument sequence, the microtiter plate was moved to a microtiter plate reader where 150 μL of ARCHITECT Trigger was injected into the wells in row H and read for 3 seconds.

In this study, a comparison of standard deviations in relation to the mean rlu values of untreated and treated samples suggest that the treatment does not result in a loss of assay precision (Table 14). Additionally, the mean values for the negative control and HBsAg 250 pg/mL sample were similar. This indicates that the treatment does not have a detrimental effect on signal.

TABLE 14
Results of Treating Replicates of Sample

| Condition D | Mean rlu | S/N | s.d. | % CV | TR:UN |
|---|---|---|---|---|---|
| Untreated Sample | | | | | |
| NC | 137 | | 19.0 | 13.9 | |
| HBsAg 250 pg/ml | 1462 | 10.7 | 76.5 | 5.2 | 1.0 |
| IC Sample | 134 | 1.0 | 17.5 | 13.0 | 4.2 |
| Treated Sample | | | | | |
| NC | 139 | | 15.2 | 11.0 | |
| HBsAg 250 pg/ml | 1451 | 10.4 | 43.8 | 3.0 | |
| IC Sample | 578 | 4.2 | 40.6 | 7.0 | |

The analytical sensitivity of the assay using 250 μL of sample volume and immune complex dissociation steps was determined by testing members of the Abbott HBsAg Sensitivity panel. This was conducted to gain information on the impact of using the low pH treatment on the assay performance. The general detection method as indicated above and as in Table 12 and Table 13 with the exception of microparticle volume (70 μL versus 50 μL) was followed.

The detectable signals measured from negative samples were used to calculate a cut-off (CO) value for the treated (i.e., with low pH) and untreated (i.e., with PBS) samples. The CO values were the mean of the negative samples, plus 10 standard deviations. For the untreated samples, the CO value was 450 (mean=174, SD=27.6), and for the treated samples, the CO value was 330 (mean=170, SD=16.0).

For the ad subtype, the analytical sensitivity of the untreated sample format was 0.044 ng/mL, and the analytical sensitivity of the treated samples was 0.013 ng/mL. In this experiment, the calculated CO for the treated samples was lower than the untreated samples and this contributed to the improved sensitivity when testing treated samples. Based on the calculated sensitivity, treatment with glycine at low pH does not appear to compromise the sensitivity of the HBsAg KF assay.

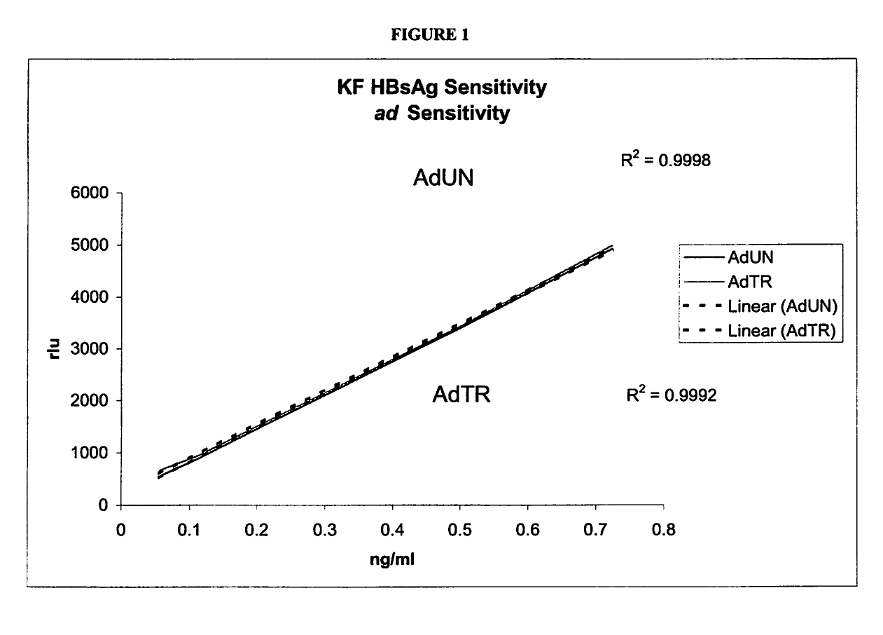

The signal or rlu values were very similar between the untreated and treated conditions (FIG. 1). Accordingly, measuring HBsAg levels upon displacement of HBsAg/anti-HBs complexes did not impair the analytical sensitivity. A HBsAg assay with improved sensitivity and with the ability to detect samples with immune complexes should lead to improved performance of the HBsAg assay and increased detection of HBV occult samples.

1. A method for detecting a target in a sample, wherein the target is bound to a first binding partner, comprising:
(a) providing a sample;
(b) displacing the target/first binding partner complex; and
(c) contacting the sample with a second binding partner,
wherein the presence of a target/second binding partner complex is indicative of the presence of the target in the sample.

2. The method of claim 1, wherein the first binding partner is an antibody.

3. The method of claim 1, wherein the second binding partner is an antibody.

4. The method of claim 1, wherein the target is a protein.

5. The method of claim 1, wherein (b) and (c) are performed concurrently.

6. The method of claim 1, further comprising detecting the target/second binding partner complex.

7. The method of claim 6, wherein (b) and (c), and the detecting are performed concurrently.

8. The method of claim 1, further comprising removing the displaced first binding partner from the sample.

9. The method of claim 8, wherein the first binding partner is removed by using an antibody binding reagent.

10. The method of claim 9, wherein the antibody binding reagent is anti-human Ig.

11. The method of claim 9, wherein the antibody binding reagent is attached to a microparticle.

12. The method of claim 4, wherein the second binding partner binds to an antigen of the protein.

13. The method of claim 12, wherein the antigen is a linear epitope.

14. The method of claim 12, wherein the antigen is a viral antigen.

15. The method of claim 14, wherein the virus is hepatitis B virus.

16. The method of claim 15, wherein the antigen is a surface antigen.

17. The method of claim 15, wherein the sample is isolated from a patient with acute or chronic hepatitis B virus infection.

18. The method of claim 15, wherein the sample is isolated from a patient with occult hepatitis B virus infection.

19. The method of claim 14, wherein the virus is hepatitis C virus.

20. The method of claim 14, wherein the virus is human immunodeficiency virus.

21. The method of claim 1, wherein the sample has a volume of at least 20 μL.

22. The method of claim 1, wherein (b) and (c) are performed on the sample in at least duplicate.

23. The method of claim 1, wherein the displacing is performed by a method selected from the group consisting of: decreasing the sample pH to 2-6, increasing the temperature of the sample to 60° C. to 110° C., and adding an agent capable of reducing disulfide bonds.

24. The method of claim 23, wherein the pH of the sample is decreased with a solution comprising glycine at a concentration of 50 mM to 1M, and wherein the pH of the solution is from 1 to 2.
